(19) United States
(12) Patent Application Publication
(10) Pub. No.: US 2021/0249138 A1
(43) Pub. Date: Aug. 12, 2021

(54) DISEASE RISK PREDICTION DEVICE, DISEASE RISK PREDICTION METHOD, AND DISEASE RISK PREDICTION PROGRAM

(71) Applicant: NEC Corporation, Minato-ku, Tokyo (JP)

(72) Inventors: Masahiro HAYASHITANI, Tokyo (JP); Masahiro KUBO, Tokyo (JP)

(73) Assignee: NEC Corporation, Minato-ku, Tokyo (JP)

(21) Appl. No.: 16/973,988

(22) PCT Filed: Jun. 6, 2019
(86) PCT No.: PCT/JP2019/022459

§ 371 Date: Dec. 10, 2020

(30) Foreign Application Priority Data
Jun. 18, 2018 (JP) 2018-115129

(51) Int. Cl. G16H 50/30; G16H 10/60; G16H 50/50; G16H 50/20; G16H 70/60; G06N 20/00

(52) U.S. Cl. CPC G16H 50/30; G16H 10/60; G06N 20/00; G16H 50/20; G16H 70/60; G16H 50/50

(57) ABSTRACT

A disease risk prediction device 80 includes a prediction unit 81 and a prediction result output unit 82. The prediction unit 81 predicts a development risk of an infectious disease using a prediction model for predicting a development status of the infectious disease, the prediction model being learned based on electronic data of a patient. The prediction result output unit 82 outputs the predicted development risk.

### TECHNICAL FIELD

The present invention relates to a disease risk prediction device, a disease risk prediction method, and a disease risk prediction program for predicting the risk of a patient developing a disease.

### BACKGROUND ART

It is a common practice to examine a patient's symptom and determine an appropriate treatment. Moreover, the risk of developing a disease is predicted based on the patient's condition. For example, Patent Literature (PTL) 1 describes a method of observing the response of a patient to a treatment selected by a doctor and the like and helping reduce the risk of developing diabetic heart disease and its complication. With the method described in PTL 1, a diabetic heart disease category risk level is assigned based on comparison between the patient's biomarker test result and a reference value range.

Non Patent Literature (NPL) 1 describes a method of predicting the risk of a patient developing a complication from accumulated time-series vital data. With the method described in NPL 1, for each of three cases of “septic shock”, “sharp blood pressure drop episode”, and “hypoxemia” which are lethal and frequent complications for intensive care unit (ICU) inpatients, a model for predicting the risk of developing the symptom 2 hours before the disease development is generated.

### CITATION LIST

### Patent Literature

PTL 1: Japanese Translation of PCT International Application Publication No. 2016-505811

### Non Patent Literature

NPL 1: NTT DATA Corporation, “Commencement of Demonstrative Experiments of Smart Alert Solution for Prevention of Complication at Medical Institution in Spain”, [online], Jan. 27, 2015 [search on May 22, 2018], Internet <URL:http://www.nttdata.com/jp/ja/news/release/2017/012701.html>.

### SUMMARY OF INVENTION

### Technical Problem

If a patient develops an infectious disease such as a complication after transferred from an ICU to a general ward, the period of hospitalization tends to prolong. There is thus a need to predict the risk of a patient developing an infectious disease in an initial stage of hospitalization.

The method described in PTL 1 is a method for reducing the risk of developing diabetic heart disease. A test is conducted according to the risk of diabetic heart disease, and a risk level is determined based on the test result. Diseases which inpatients may develop are, however, not limited to one specific disease such as diabetic heart disease. It is impractical to conduct tests for all possible diseases.

The method described in NPL 1 is limited to ICU patients. Specifically, the method described in NPL 1 requires a lot of information for generating a model, including information of not only a vital signs monitor but also a ventilator, an infusion pump, and the like.

Typically, in an initial stage of hospitalization, a set of examination data is collected, and accumulated as information of an electronic medical record. It is therefore preferable to predict, from such data collected in the initial stage of hospitalization, the risk of developing an infectious disease subsequently.

The present invention accordingly has an object of providing a disease risk prediction device, a disease risk prediction method, and a disease risk prediction program that can predict an inpatient's risk of an infectious disease.

### Solution to Problem

A disease risk prediction device according to the present invention includes: a prediction unit for predicting a development risk of an infectious disease using a prediction model for predicting a development status of the infectious disease, the prediction model being learned based on electronic data of a patient; and a prediction result output unit for outputting the predicted development risk.

A disease risk prediction method according to the present invention includes: predicting a development risk of an infectious disease using a prediction model for predicting a development status of the infectious disease, the prediction model being learned based on electronic data of a patient; and outputting the predicted development risk.

A disease risk prediction program according to the present invention causes a computer to execute: a prediction process of predicting a development risk of an infectious disease using a prediction model for predicting a development status of the infectious disease, the prediction model being learned based on electronic data of a patient; and a prediction result output process of outputting the predicted development risk.

### Advantageous Effects of Invention

According to the present invention, an inpatient's risk of an infectious disease can be predicted.

### BRIEF DESCRIPTION OF DRAWINGS

FIG. 1 It is a block diagram depicting an example of a structure of an exemplary embodiment of a disease risk prediction device according to the present invention.

FIG. 2 It is an explanatory diagram depicting an example of items included in an electronic medical record.

FIG. 3 It is an explanatory diagram depicting an example of output evaluation results.

FIG. 4 It is a flowchart depicting an example of operation of the disease risk prediction device.

FIG. 5 It is a block diagram depicting an overview of a disease risk prediction device according to the present invention.

FIG. 6 It is a schematic block diagram depicting a structure of a computer according to at least one exemplary embodiment.

### DESCRIPTION OF EMBODIMENT

An exemplary embodiment of the present invention will be described below, with reference to the drawings.

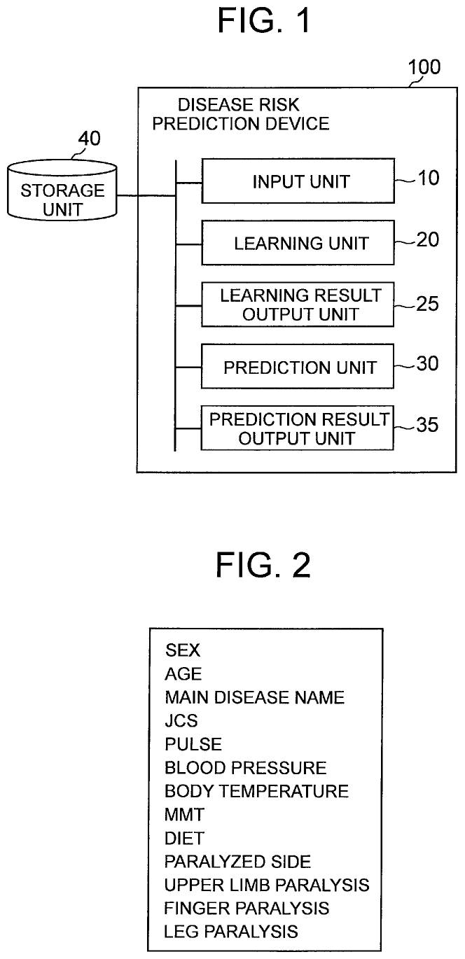

FIG. 1 is a block diagram depicting an example of a structure of an exemplary embodiment of a disease risk prediction device according to the present invention. A disease risk prediction device 100 in this exemplary embodiment includes an input unit 10, a learning unit 20, a learning result output unit 25, a prediction unit 30, and a prediction result output unit 35. The disease risk prediction device 100 is connected to a storage unit 40. The storage unit 40 may be located in any state. For example, the storage unit 40 may be a storage in cloud computing, or may be included in the disease risk prediction device 100.

The storage unit 40 stores various types of information (hereafter referred to as “patient information”) of a patient. Typically, when a patient is hospitalized, various examinations are conducted to collect patient information. Accordingly, the patient information is recorded in the patient's electronic medical record one to two days after admission. The storage unit 40 stores this electronic medical record. For example, the patient information includes not only the examination data but also the patient's profile, background information, etc.

FIG. 2 is an explanatory diagram depicting an example of items included in an electronic medical record. For example, information of the items depicted in FIG. 2 are collected in an initial stage of hospitalization, and stored in the storage unit 40 as patient information. The items depicted in FIG. 2 may include information that changes in chronological order. For example, the pulse, blood pressure, body temperature, etc. may be stored in chronological order upon each examination.

The input unit 10 reads the electronic data of the patient from the storage unit 40, and inputs the electronic data to the learning unit 20 and the prediction unit 30. The below-described learning unit 20 learns a prediction model based on the input information. The below-described prediction unit 30 predicts the risk of developing an infectious disease based on the input information.

The learning unit 20 learns a prediction model for predicting the development status of the infectious disease by machine learning using, as training data, data associating the electronic data of the patient and the development status of the infectious disease of the patient. Specifically, the electronic data of the patient is information of the electronic medical record stored in the storage unit 40. For example, the electronic data of the patient is patient information including the items depicted in FIG. 2. These items are used as explanatory variables of the prediction model. Any method may be used by the learning unit 20 to learn the prediction model. A widely known method may be used.

In the following description, the degree representing the development status of the infectious disease to be predicted is referred to as “development risk”. The development risk may be expressed in any way, and is determined depending on the prediction model generated as a result of learning. As an example, in the case where a prediction model for predicting, as the development status, whether or not development occurs is learned, the development risk is expressed as “whether or not there is possibility of development”. As another example, in the case where a prediction model for predicting the likelihood of development is learned, the development risk is expressed as “development risk: ◯◯percent”. As another example, in the case where a prediction model for predicting, as the development status, the stage of development, the development risk is expressed in stages (e.g. “level ◯”).

This exemplary embodiment describes aspiration pneumonia as a specific example of the infectious disease. Aspiration pneumonia is a complication and also a kind of infectious disease, and is a disease that not only occurs alone but also occurs frequently as a complication of stroke patients. In the case of learning a prediction model for predicting the development status of aspiration pneumonia, the learning unit 20 preferably uses training data including the patient's consciousness level and body temperature. Examples of an index for evaluating the patient's consciousness level include Japan Coma Scale (JCS) and Glasgow Coma Scale (GCS). JCS and GCS are each used, for example, as an index for evaluating the consciousness level of a patient with impaired consciousness.

Furthermore, given that the consciousness level and the body temperature are likely to change in chronological order, the learning unit 20 may learn, for example, a prediction model for predicting the development risk of aspiration pneumonia using training data including the patient's time-series consciousness level and body temperature. For example, an explanatory variable representing JCS on the first day of hospitalization may be denoted by x1, an explanatory variable representing JCS on the second day of hospitalization by x2, and so on.

Due to the nature of machine learning, there is a possibility that, even when the same training data and the same algorithm are used, a different prediction model is generated depending on the initial value. Hence, the learning unit 20 may generate a plurality of types of prediction models using the same training data and the same algorithm.

In a hospital treating acutely ill patients, discharge of a patient from hospital in two weeks is considered appropriate. If the patient develops a complication about one week after admission, however, the hospitalization tends to prolong (e.g. for one month). It is therefore preferable to predict, from the patient's condition in the initial stage of hospitalization, the development risk of the infectious disease. Thus, the learning unit 20 preferably learns the prediction model based on the patient information collected in the initial stage of hospitalization.

The learning result output unit 25 outputs the learning result of the prediction model. The learning result output unit 25 may display the learned prediction model itself, or output a result of evaluating the prediction model. For example, in the case where the prediction model is expressed by a formula using explanatory variables, the learning result output unit 25 may output the formula expressing the prediction model as a prediction formula.

Suppose the prediction model includes, as an element, a branch condition (gate function) for selecting a component assigned depending on an input sample, as in a prediction model generated by a heterogeneous mixture learning algorithm. In this case, the learning result output unit 25 may output the prediction model in the form of a binary tree structure (specifically, a structure in which leaf nodes are components and other upper nodes are gate functions).

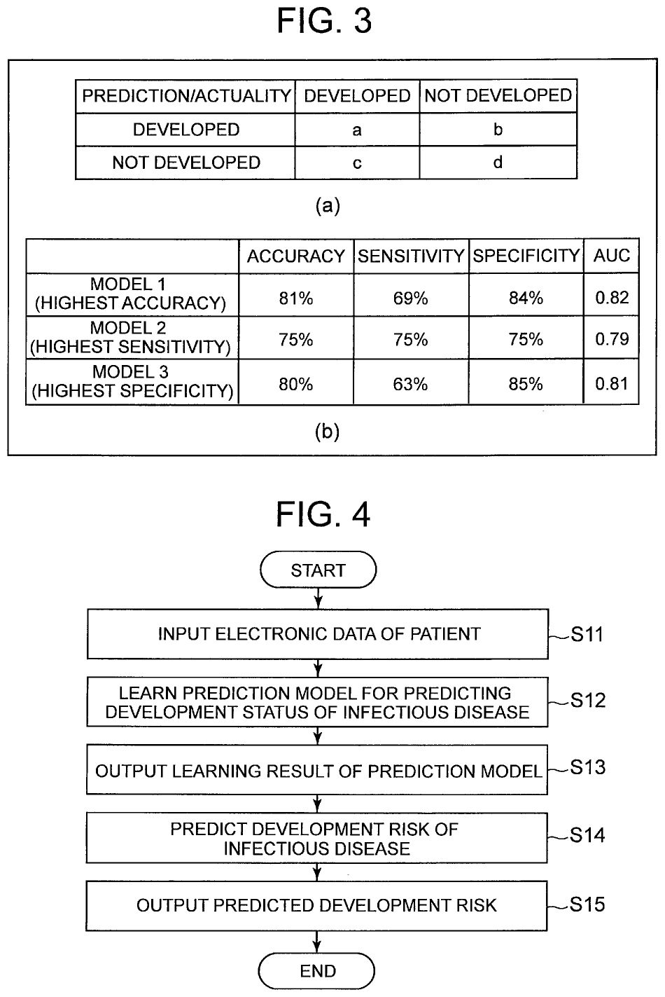

The learning result output unit 25 may output, for each generated prediction model, a result based on predetermined evaluation using, as evaluation data, data associating the patient information and the development status of the infectious disease of the patient. FIG. 3 is an explanatory diagram depicting an example of output evaluation results. As depicted in FIG. 3(a), for example, accuracy (a/(a+b)), sensitivity (a/(a+c)), specificity (d/(b+d)), AUC (area under the curve), and the like can be used for evaluation.

The learning result output unit 25 may output, for example, a plurality of models for each evaluation result, as depicted in FIG. 3(b). In the example depicted in FIG. 3(b), model 1 has the highest accuracy of the three models, model 2 has the highest sensitivity of the three models, and model 3 has the highest specificity of the three models.

The learning result output unit 25 may receive, from a user, selection of a prediction model used by the below-described prediction unit 30. As a result of the selection of the prediction model being received from the user, the below-described prediction unit 30 can perform prediction from a viewpoint on which the user places importance.

The prediction unit 30 predicts the development risk of the infectious disease using the prediction model. Specifically, the prediction unit 30 predicts the development risk of the infectious disease using the prediction model generated by the learning unit 20. The prediction unit 30 may use a prediction model selected by the user, or a prediction model corresponding to an evaluation result that satisfies a predetermined standard level. To prevent overlooking the signs of disease development, the prediction unit 30 may select a prediction model having the highest sensitivity.

In the case where there are a plurality of prediction models, the prediction unit 30 may predict the development risk of the infectious disease using the plurality of prediction models. For example, in the case where there are the foregoing three types of prediction models, the prediction unit 30 may predict the development risk of the infectious disease using all of the prediction models. As a result of the prediction unit 30 predicting the development risk of the infectious disease using the plurality of prediction models, for example, a doctor can take measures or it makes it possible to ask for a doctor in the case where all prediction models predict high development risk.

It is recognized that countermeasures to complications for stroke patients significantly contribute to early hospital discharge. Aspiration pneumonia is known to be a frequent complication for stroke patients, as mentioned above. As a result of the prediction unit 30 applying patient information of a stroke patient to the prediction model, the risk of the stroke patient developing aspiration pneumonia can be promptly predicted.

The prediction result output unit 35 outputs the development risk predicted by the prediction unit 30. The expression of the development risk depends on the prediction model. For example, the prediction result output unit 35 may output the development risk expressed as “whether or not there is possibility of development”, “development risk: ◯◯ percent”, or “level ◯”. As a result of the prediction result output unit 35 outputting the predicted development risk, which inpatient requires particular attention can be identified. In the case where the prediction unit 30 predicts the development risk of the infectious disease using a plurality of prediction models, the prediction result output unit 35 may output the prediction result of each prediction model.

The prediction result output unit 35 may output information of which explanation variable is effective in the prediction from among the explanatory variables included in the prediction model used for the prediction. For example, in the case where the prediction model is represented by a linear combination of explanatory variables, the coefficient of each explanatory variable corresponds to the weight of the explanatory variable for the prediction result. Accordingly, the prediction result output unit 35 may output the weight of each explanatory variable included in the prediction model in the form of a bar graph.

The prediction result output unit 35 may output each of the prediction model and the patient information used in the prediction, and output information of which explanatory variable is effective in the prediction of the development risk. For example, in the case where the development risk of patient A is predicted, the prediction result output unit 35 may output information that patient A has higher body temperature than the standard level and has low consciousness level from among the explanatory variables.

The input unit 10, the learning unit 20, the learning result output unit 25, the prediction unit 30, and the prediction result output unit 35 are implemented by a central processing unit (CPU) in a computer operating according to a program (disease risk prediction program). For example, the program may be stored in a storage unit (not depicted) included in the disease risk prediction device 100, with the CPU reading the program and, according to the program, operating as the input unit 10, the learning unit 20, the learning result output unit 25, the prediction unit 30, and the prediction result output unit 35.

The input unit 10, the learning unit 20, the learning result output unit 25, the prediction unit 30, and the prediction result output unit 35 may each be implemented by dedicated hardware.

Operation of the disease risk prediction device in this exemplary embodiment will be described below. FIG. 4 is a flowchart depicting an example of operation of the disease risk prediction device in this exemplary embodiment.

The input unit 10 reads the electronic medical record stored in the storage unit 40, and inputs the electronic data of the patient to the learning unit 20 and the prediction unit 30 (step S11). The learning unit 20 learns a prediction model for predicting the development status of the infectious disease based on the input electronic data of the patient (step S12). The learning result output unit 25 outputs the learning result of the prediction model (step S13). In the case where the prediction model has already been learned, the processes in steps S12 and S13 may be omitted.

The prediction unit 30 predicts the development risk of the infectious disease using the prediction model (step S14). The prediction unit 30 may predict the development risk of the infectious disease using a plurality of prediction models. The prediction result output unit 35 then outputs the predicted development risk (step S15). In the case where the development risk of the infectious disease is predicted using the plurality of prediction models as mentioned above, the prediction result output unit 35 may output the development risk predicted for each prediction model.

As described above, in this exemplary embodiment, the prediction unit 30 predicts the development risk of the infectious disease using the prediction model learned based on the electronic data of the patient, and the prediction result output unit 35 outputs the predicted development risk. Thus, the inpatient's risk of the infectious disease can be predicted. Moreover, by the learning unit 20 learning the prediction model by machine learning using appropriately selected data, the prediction accuracy of the prediction model can be improved while reducing the computer processing load.

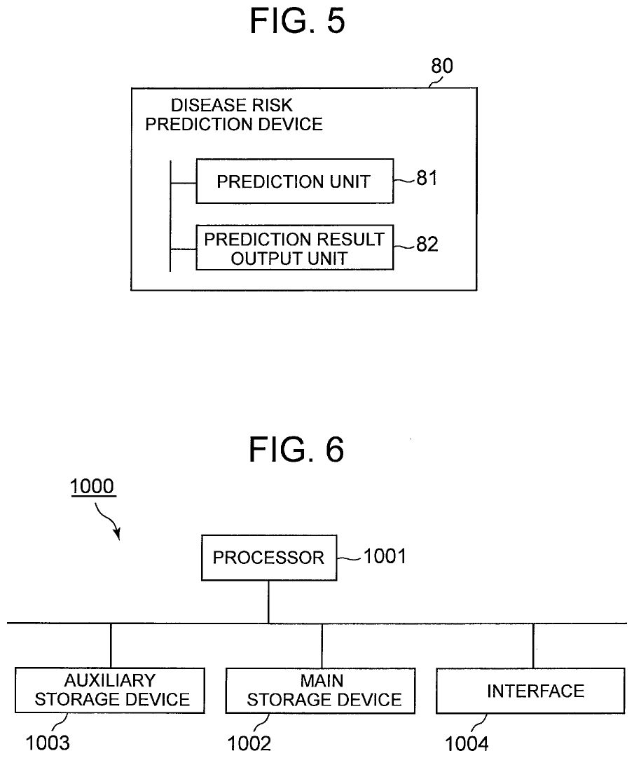

An overview of the present invention will be described below. FIG. 5 is a block diagram depicting an overview of a disease risk prediction device according to the present invention. A disease risk prediction device 80 (e.g. disease risk prediction device 100) includes: a prediction unit 81 (e.g. prediction unit 30) for predicting a development risk of an infectious disease using a prediction model for predicting a development status of the infectious disease, the prediction model being learned based on electronic data (e.g. electronic medical record) of a patient; and a prediction result output unit 82 (e.g. prediction result output unit 35) for outputting the predicted development risk.

With such a structure, the inpatient's risk of the infectious disease can be predicted.

The disease risk prediction device 80 may include a learning unit (e.g. learning unit 20) for learning the prediction model for predicting the development status of the infectious disease, based on the electronic data of the patient, and the prediction unit 81 may predict the development risk of the infectious disease using the prediction model learned by the learning unit.

Specifically, the learning unit may learn the prediction model having consciousness level (e.g. JCS, GCS, or the like) and body temperature as explanatory variables, and the prediction unit 81 may predict the development risk of the infectious disease using the prediction model. The learning unit may learn the prediction model for predicting a development risk of aspiration pneumonia, using training data including consciousness level and body temperature of the patient. By using such explanatory variables effective in the prediction of the development risk of aspiration pneumonia, the accuracy of the prediction model can be improved.

The learning unit may learn the prediction model for predicting a development risk of aspiration pneumonia, using training data including time-series consciousness level and body temperature of the patient. With such a structure, the prediction model can be learned based on the patient's condition that changes on a daily basis.

The disease risk prediction device 80 may include a learning result output unit (e.g. learning result output unit 25) for outputting the learned prediction model, the learning unit may generate a plurality of types of prediction models based on the electronic data of the patient, and the learning result output unit may output, for each generated prediction model, a result based on predetermined evaluation (e.g. accuracy, sensitivity, specificity, AUC, etc.), using, as evaluation data, data associating the electronic data of the patient and the development status of the infectious disease of the patient. With such a structure, a prediction model that is evaluated high from a viewpoint on which the user places importance can be selected.

FIG. 6 is a schematic block diagram depicting a structure of a computer according to at least one exemplary embodiment. A computer 1000 includes a processor 1001, a main storage device 1002, an auxiliary storage device 1003, and an interface 1004.

The disease risk prediction device described above is implemented by the computer 1000. The operation of each processing unit described above is stored in the auxiliary storage device 1003 in the form of a program (disease risk prediction program). The processor 1001 reads the program from the auxiliary storage device 1003, expands the program in the main storage device 1002, and executes the above-described process according to the program.

In at least one exemplary embodiment, the auxiliary storage device 1003 is an example of a non-transitory tangible medium. Examples of the non-transitory tangible medium include a magnetic disk, magneto-optical disk, CD-ROM (compact disc read-only memory), DVD-ROM (read-only memory), and semiconductor memory connected via the interface 1004. In the case where the program is distributed to the computer 1000 through a communication line, the computer 1000 to which the program has been distributed may expand the program in the main storage device 1002 and execute the above-described process.

The program may realize part of the above-described functions. The program may be a differential file (differential program) that realizes the above-described functions in combination with another program already stored in the auxiliary storage device 1003.

Although the present invention has been described with reference to the exemplary embodiments and examples, the present invention is not limited to the foregoing exemplary embodiments and examples. Various changes understandable by those skilled in the art can be made to the structures and details of the present invention within the scope of the present invention.

This application claims priority based on Japanese Patent Application No. 2018-115129 filed on Jun. 18, 2018, the disclosure of which is incorporated herein in its entirety.

### REFERENCE SIGNS LIST

10 input unit
20 learning unit
25 learning result output unit
30 prediction unit
35 prediction result output unit
40 storage unit
100 disease risk prediction device

What is claimed is:

1. A disease risk prediction device, comprising a hardware processor configured to execute a software code to:
predict a development risk of an infectious disease using a prediction model for predicting a development status of the infectious disease, the prediction model being learned based on electronic data of a patient; and
output the predicted development risk.

2. The disease risk prediction device according to claim 1, wherein the hardware processor is configured to execute a software code to:
learn the prediction model for predicting the development status of the infectious disease, based on the electronic data of the patient; and
predict the development risk of the infectious disease using the learned prediction model.

3. The disease risk prediction device according to claim 2, wherein the hardware processor is configured to execute a software code to:
learn the prediction model having consciousness level and body temperature as explanatory variables; and
predict the development risk of the infectious disease using the prediction model.

4. The disease risk prediction device according to claim 2, wherein the hardware processor is configured to execute a software code to learn the prediction model for predicting a development risk of aspiration pneumonia, using training data including consciousness level and body temperature of the patient.

5. The disease risk prediction device according to claim 2, wherein the hardware processor is configured to execute a software code to learn the prediction model for predicting a development risk of aspiration pneumonia, using training data including time-series consciousness level and body temperature of the patient.

6. The disease risk prediction device according to claim 2, wherein the hardware processor is configured to execute a software code to:
generate a plurality of types of prediction models based on the electronic data of the patient; and
output, for each generated prediction model, a result based on predetermined evaluation using, as evaluation data, data associating the electronic data of the patient and the development status of the infectious disease of the patient.

7. A disease risk prediction method, comprising:
predicting a development risk of an infectious disease using a prediction model for predicting a development status of the infectious disease, the prediction model being learned based on electronic data of a patient; and
outputting the predicted development risk.

8. A non-transitory computer readable information recording medium storing a disease risk prediction program, when executed by a processor, that performs a method for:
predicting a development risk of an infectious disease using a prediction model for predicting a development status of the infectious disease, the prediction model being learned based on electronic data of a patient; and
outputting the predicted development risk.
